(19) United States
(12) Patent Application Publication
(10) Pub. No.: US 2016/0122281 A1
(43) Pub. Date: May 5, 2016

(54) PROCESS FOR PRODUCING AROMATIC CARBONATES

(71) Applicant: SHELL OIL COMPANY, Houston, TX (US)

(72) Inventor: Garo Garbis VAPORCIYAN, Houston, TX (US)

(21) Appl. No.: 14/892,306

(22) PCT Filed: May 20, 2014
(86) PCT No.: PCT/US2014/038706

§ 371 Date: Nov. 19, 2015

Related U.S. Application Data
(60) Provisional application No. 61/826,101, filed on May 22, 2013.

(51) Int. Cl. C07C 68/06; C07C 68/08; C07C 29/00

(52) U.S. Cl. CPC C07C 68/06; C07C 29/00; C07C 68/08

(57) ABSTRACT

This invention provides a method for producing an alkylaryl carbonate comprising: a) contacting a stream comprising an aromatic hydroxy compound and a stream comprising a dialkylcarbonate in the presence of a transesterification catalyst in a reactive distillation column; b) withdrawing a first product stream comprising the alkylaryl carbonate from the reactive distillation column; c) withdrawing a second product stream comprising alkyl hydroxy compound and dialkylcarbonate; d) adding fresh dialkylcarbonate to the second product stream; e) separating the dialkylcarbonate from the alkyl hydroxy compound; and f) recycling the dialkylcarbonate from step e) to the reactive distillation column.

The present application claims the benefit of pending U.S. Provisional Patent Application Ser. No. 61826101, filed May 22, 2013.

### FIELD OF THE INVENTION

This invention relates to the production of aromatic carbonates.

### BACKGROUND OF THE INVENTION

Aromatic carbonates are typically produced by a transesterification reaction between a dialkylcarbonate and an aromatic hydroxy compound. This reaction is typically carried out in the presence of a catalyst to accelerate the transesterification reaction. The catalyst may be homogeneous and/or heterogeneous. Aromatic carbonates are useful as raw materials for the production of aromatic polycarbonates that are used as engineering plastics.

U.S. Pat. No. 5,334,742 describes a process for preparing diarylcarbonates by reacting dialkylcarbonates with phenol using conventional transesterification catalysts in a specific mass-coupled and energy-coupled combination of columns. The WO 01/00560 publication describes a process for preparing aromatic carbonates by gas phase reaction or liquid phase reaction of dimethylcarbonate with phenol in the presence of a titanium-silica catalyst followed by the liquid phase reaction of the prepared methylphenylcarbonate in the presence of a titanium-silica catalyst to produce the aromatic carbonates.

### SUMMARY OF THE INVENTION

The invention provides a method for producing an alkylaryl carbonate comprising: a) contacting a stream comprising an aromatic hydroxy compound and a stream comprising a dialkylcarbonate in the presence of a transesterification catalyst in a reactive distillation column; b) withdrawing a first product stream comprising the alkylaryl carbonate from the reactive distillation column; c) withdrawing a second product stream comprising alkyl hydroxy compound and dialkylcarbonate; d) adding fresh dialkylcarbonate to the second product stream; e) separating the dialkylcarbonate from the alkyl hydroxy compound; and f) recycling the dialkylcarbonate from step e) to the reactive distillation column.

### BRIEF DESCRIPTION OF THE DRAWINGS

FIG. 1 depicts an apparatus for the production of aromatic carbonates.

### DETAILED DESCRIPTION

The process for producing aromatic carbonates involves the transesterification of dialkylcarbonates and aromatic hydroxy compounds. The aromatic carbonates produced are typically in the form of alkylarylcarbonates, although diarylcarbonates can be formed through a subsequent disproportionation reaction. The aromatic carbonates produced in the reactive distillation column may be alkylarylcarbonates, diarylcarbonates or a mixture thereof.

The dialkylcarbonate is represented by the formula R¹OCOOR¹. R¹ represents an alkyl group having 1 to 10 carbon atoms, an alicyclic group having 3 to 10 carbon atoms or an aralkyl group having 6 to 10 carbon atoms. Examples of R¹ include an alkyl group, such as methyl, ethyl, propyl, allyl, butyl, butenyl, pentyl, hexyl, heptyl, octyl, nonyl, decyl and cyclohexylmethyl and isomers thereof. Further examples of R¹ include an alicyclic group, such as cyclopropyl, cyclobutyl, cyclopentyl, cyclohexyl, and cycloheptyl; and an aralkyl group, such as benzyl, phenethyl, phenylpropyl, phenylbutyl, methylbenzyl and isomers thereof.

The alkyl, alicyclic or aralkyl group may be substituted with a substituent such as a lower alkyl group, a lower alkoxy group, a cyano group and a halogen atom.

Examples of the dialkylcarbonate are dimethylcarbonate, diethylcarbonate, dipropylcarbonate, diallylcarbonate, dibutenylcarbonate, dibutylcarbonate, dipentylcarbonate, dihexylcarbonate, diheptylcarbonate, dioctylcarbonate, dinonylcarbonate, didecylcarbonate, dicyclopentylcarbonate, dicyclohexylcarbonate, dicycloheptylcarbonate, and isomers thereof.

A dialkylcarbonate where R¹ is an alkyl group having four or less carbon atoms is preferred. The dialkylcarbonate is most preferably diethylcarbonate.

The aromatic hydroxy compound is represented by the formula Ar¹ OH where Ar¹ represents an aromatic group having 5 to 30 carbon atoms, and the type of compound is not limited as long as the hydroxy group is directly bonded to the aromatic group. Examples of Ar¹ include a phenyl group and various alkylphenyl groups, such as, tolyl, xylyl, trimethylphenyl, tetramethylphenyl, ethylphenyl, propylphenyl, butylphenyl, diethylphenyl, methylethylphenyl, pentylphenyl, hexylphenyl, cyclohexylphenyl, and isomers thereof; various alkoxyphenyl groups, such as, methoxyphenyl ethoxyphenyl, butoxyphenyl and isomers thereof; various halogenated phenyl groups, such as fluorophenyl, chlorophenyl, bromophenyl, chloromethylphenyl, dichlorophenyl, and isomers thereof.

Examples of aromatic hydroxy compounds having these Ar¹ include phenol; various alkyl phenols, such as cresol, xylenol, trimethylphenol, tetramethylphenol, ethylphenol, propylphenol, butylphenol, diethylphenol, methylethylphenol, methylpropylphenol, dipropylphenol, methylbutylphenol, pentylphenol, hexylphenol and cyclohexylphenol; various alkoxyphenols, such as methoxyphenol and ethoxyphenol; and isomers thereof. An aromatic monohydroxy compound where Ar¹ is an aromatic group having from 6 to 10 carbon atoms is preferred and phenol is most preferred.

The transesterification reaction produces an alkylarylcarbonate corresponding to the reactants fed to the reactive distillation column and an alkyl hydroxy compound. In one embodiment, the transesterification reaction is carried out with phenol and diethylcarbonate and the resulting products are ethylphenylcarbonate and ethanol. In another embodiment where the reaction is carried out with phenol and dimethylcarbonate, the resulting transesterification products will be methylphenylcarbonate and methanol.

The transesterification reaction is an equilibrium reaction, and the equilibrium is biased toward the reactants. In addition, the reaction rate is low. To help shift the equilibrium to produce more aromatic carbonates, the reaction is carried out in a reactive distillation column. The reactive distillation column is operated so that the transesterification products are removed in the overhead product stream.

A second equilibrium reaction that occurs in the reactive distillation column is the disproportionation reaction. This reaction occurs when two alkylarylcarbonate molecules disproportionate and form a diarylcarbonate and a dialkylcarbonate. In the embodiment where ethylphenylcarbonate and ethanol are formed by transesterification, the products of the disproportionation reaction would be diphenylcarbonate and diethylcarbonate. In the embodiment where methylphenylcarbonate and methanol are formed, the products of the disproportionation reaction would be diphenylcarbonate and dimethylcarbonate.

The products of the transesterification reaction and/or the disproportionation reaction are removed from the reactive distillation column at one or more outlets and separated and/or recycled to the reactive distillation column or other process units.

The reactive distillation column may contain any internals known to one of ordinary skill in the art to be useful in a reactive distillation column. Examples of suitable columns include plate type columns using a tray, such as a bubble-cap tray, a sieve tray, a valve tray, and a counterflow tray; and packed type columns packed with various packings, such as Raschig ring, a Lessing ring, a Pall ring, a Berl saddle, an Intelox saddle, a Dixon packing, a McMahon packing, a Heli pack, a Sulzer packing and Mellapak.

The heterogeneous catalyst used in this reactive distillation column may be any catalyst known to one of ordinary skill in the art to be useful in accelerating the transesterification reaction. The heterogeneous catalyst may comprise titanium, chromium, tungsten, molybdenum, vanadium, tin, lead, copper, alkali metals, zinc, cadmium, iron, zirconium, Lewis Acid, Lewis Acid-forming compounds or a mixture thereof. The catalyst preferably comprises titanium.

The heterogeneous catalyst may be supported on aluminium oxide, titanium oxide, silicon oxide, active carbon or a mixture thereof. The catalyst is preferably supported on silica. The catalyst is preferably titanium supported on silica.

Further, a homogenous catalyst may be added to the reaction with or without a heterogeneous catalyst or support already present in the reactive distillation column. In one embodiment, the homogenous catalyst may be added to replace metals that are leached from the heterogeneous catalyst. The homogeneous catalyst preferably comprises titanium-ethanolate, titanium-phenolate. The homogeneous catalyst may be fed in a solution of phenol.

The homogeneous catalyst may be added to maintain a specific concentration of metals in the reactive distillation column. The concentration of metals in the column may be monitored by measuring the level of metals in the bottom product stream. The concentration of metals may be in the range of from 10 to 2000 milligram of metal per kilogram of product stream, preferably of from 50 to 250 mg/kg, and more preferably of from 80 to 200 mg/kg.

One of the difficulties encountered in this reaction is that the heterogeneous and homogeneous catalysts are deactivated by contact with catalyst poisons, e.g., water. This description will focus on water as a catalyst poison, but any poison that boils below the boiling point of the dialkylcarbonate can be removed from the dialkylcarbonate by this method. The dialkyl carbonate compound used as a reactant in this process may contain water. Even when purified to remove the water a residual amount of water may be present in the dialkyl carbonate stream. Water may be present in the feed stream comprising the dialkyl carbonate compound in an amount of up to 0.5 wt %, but is preferably only present in an amount of less than 1000 ppmw, more preferably less than 300 ppmw and most preferably less than 150 ppmw.

Some possible methods to remove this water include separating the water in a separate distillation column, and using an adsorbent or absorbent. The use of a dedicated column would result in increased cost and energy use. In addition, it is difficult to find adsorbents or absorbents which can achieve the desired water level but do not leach substances that would have a detrimental effect on the catalyst or process.

The invention provides a method of operating the process to reduce the amount of water that contacts the catalyst in the column without requiring an additional separation of water from the dialkyl carbonate feed that would be expensive or difficult.

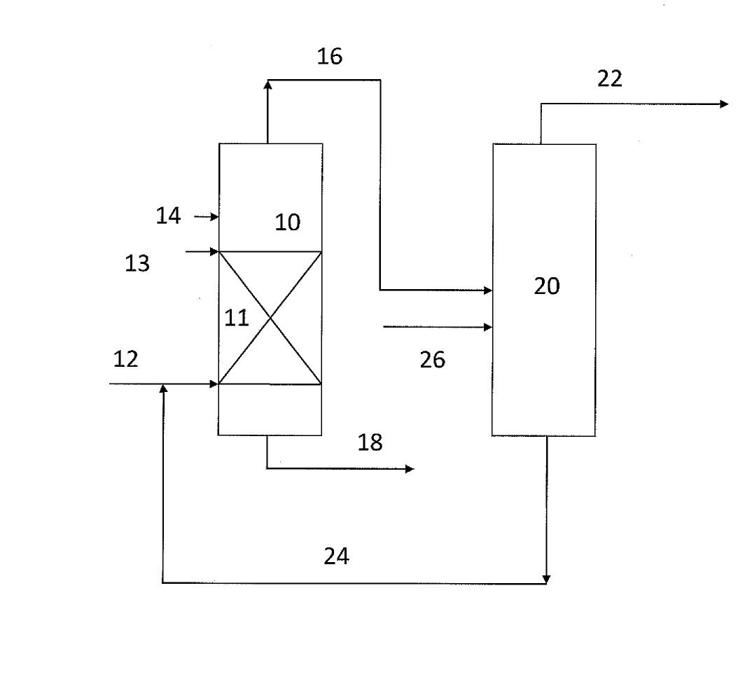

The method and the apparatus used to carry out the method will be further described in relation to FIG. 1. It is understood that one of ordinary skill in the art can modify the apparatus and method depicted in FIG. 1 while still carrying out the invention as described and as claimed hereinafter. The Figure does not depict every piece of equipment that would be used in the process including reboilers, condensers, heat exchanges, valves and pumps, but one of ordinary skill in the art could determine where to place these items in the process.

FIG. 1 depicts a reactive distillation column 10 for carrying out a transesterification reaction of a dialkylcarbonate and an aromatic hydroxy compound. A heterogeneous catalyst bed 11 may be located inside the reactive distillation column As described above, the method may, in another embodiment, be carried out without a bed of heterogeneous catalyst. The reactive distillation column has an inlet 12 for the feed comprising the aromatic hydroxy compound and the dialkylcarbonate. These may be fed at the same point or at different points in the column. In one embodiment the aromatic hydroxy compound is fed above the heterogeneous catalyst bed via optional inlet 14. The inlet 14 also serves as an optional inlet for homogeneous catalyst. In another embodiment, the homogeneous catalyst may be fed into the reactive distillation column through inlet 13.

The reactive distillation column has an outlet 16 for an overhead product stream that typically comprises a dialkylcarbonate, and an alkyl hydroxy compound. The column also has an outlet 18 for a bottom product stream that typically comprises an aromatic hydroxy compound, a dialkylcarbonate, an alkylarylcarbonate, and diarylcarbonate. Either of the outlets may contain by-products formed during the reaction.

The reactive distillation column is operated under reaction conditions that are conducive to the transesterification reaction. These conditions result in a separation of any water from the stream comprising the aromatic hydroxy compound. When the aromatic hydroxy compound is fed in inlet 14 (and the optional homogeneous catalyst via inlet 13), the water is removed from the stream before the stream contacts the heterogeneous or homogeneous catalyst, preventing the deactivation of the catalyst.

The column is typically operated at a pressure in the range of from 1 bara to 5 bara, preferably in a range of from 2 to 4 bara. The column is typically operated such that the temperature in the heterogeneous catalyst bed is in a range of from 100° C. to 250° C., preferably in a range of from 150° C. to 230° C. and more preferably in a range of from 170° C. to 210° C.

The column preferably contains some type of internals between the top of the catalyst bed 11 and the outlet 16, for example, trays, packing, Pall rings, Raschig rings or other internals known to one of ordinary skill in the art including those described previously. The internals assist in the separation of the water from the aromatic hydroxy compound.

The process also comprises a separation vessel/column 20 that is used to separate the dialkylcarbonate from the other overhead products. The dialkyl carbonate is separated and recycled via line 24 to the reactive distillation column. Fresh dialkyl carbonate that is fed into the process is fed into the separation vessel via inlet 26 or optionally together with line 16. The fresh dialkyl carbonate may be fed at the same or a different stage or, if present, tray as line 16. If fed at a different stage or tray it may be fed at a lower or higher stage or tray. In this way, water present in the dialkyl carbonate is separated in this separation vessel and the water goes overhead with the other overhead products via line 22 including the alkyl hydroxy compound present in line 16. The alkyl hydroxy compound and the water may be recycled to a process for producing alkyl carbonate.

The effectiveness of the process can be evaluated by determining the amount of water present in the heterogeneous catalyst bed. This can be an absolute measure, and in this case the amount of water in the catalyst bed is preferably less than 250 ppmw calculated against the total amount of aromatic hydroxy compound present in the catalyst bed. The amount of water in the catalyst bed is more preferably less than 150 ppmw, and most preferably less than 100 ppmw.

Another measure of the effectiveness of the process can be a relative measure, and in this case the amount of water present in the aromatic hydroxy compound when it passes into the catalyst bed is less than 80% of the amount of water in the aromatic hydroxy compound before it enters the reactive distillation column. The amount of water present in the aromatic hydroxy compound as it enters the catalyst bed is preferably less than 60% and more preferably less than 40% of the amount of water in the aromatic hydroxy compound before it enters the reactive distillation column.

The water may be removed through outlet 22 along with the overhead products. The water is typically present in the stream comprising the alkyl hydroxy compound. This stream may be recycled to a unit that produces dialkylcarbonate. The water may be separated from the alkyl hydroxy compound prior to recycling the stream. Alternatively the water may be left in the stream as it does not have a negative effect on the operation of the dialkylcarbonate production unit.

1. A method for producing an alkylaryl carbonate comprising:
a) contacting a stream comprising an aromatic hydroxy compound and a stream comprising a dialkylcarbonate in the presence of a transesterification catalyst in a reactive distillation column;
b) withdrawing a first product stream comprising the alkylaryl carbonate from the reactive distillation column;
c) withdrawing a second product stream comprising alkyl hydroxy compound and dialkylcarbonate from the reactive distillation column;
d) adding fresh dialkylcarbonate to the second product stream;
e) separating dialkylcarbonate from the alkyl hydroxy compound; and
f) recycling the dialkylcarbonate from step e) to the reactive distillation column.

2. The method of claim 1 wherein the transesterification catalyst is selected from the group consisting of a homogeneous catalyst, a heterogeneous catalyst and mixtures thereof.

3. The method of claim 1 wherein the heterogeneous catalyst is contained in a bed in the reactive distillation column.

4. The method of claim 1 wherein step (e) is carried out in a distillation column and the fresh dialkylcarbonate is added to the second product stream before the second product stream enters the distillation column.

5. The method of claim 1 wherein step (e) is carried out in a distillation column and the fresh dialkylcarbonate is added to the distillation column separately from the second product stream.

6. The method of claim 1 wherein the fresh dialkylcarbonate contains one or more catalyst poisons that boil below the boiling point of the dialkylcarbonate.

7. The method of claim 1 wherein at least one of the catalyst poisons is water.

8. The method of claim 1 wherein substantially no fresh dialkylcarbonate is added directly to the reactive distillation column.

9. The method of claim 1 wherein the water from the fresh dialkylcarbonate is separated from the fresh dialkylcarbonate in step e).

10. The method of claim 9 wherein the water is combined with the alkyl hydroxy compound from the second product stream.

11. The method of claim 10 further comprising using the water and alkyl hydroxy compound in a process to prepare an dialkylcarbonate by reaction of the alkyl hydroxy compound with an alkylcarbonate.

12. method of claim 11 further comprising producing monoethylene glycol.

13. The method of claim 1 wherein the aromatic hydroxy compound is phenol.

14. The method of claim 1 wherein the dialkylcarbonate is selected from the group consisting of dimethyl carbonate, diethyl carbonate and mixtures thereof.

15. The method of claim 1 wherein the catalyst comprises titanium.
